(19) United States
(12) Patent Application Publication
(10) Pub. No.: US 2008/0132672 A1
(43) Pub. Date: Jun. 5, 2008

(54) Composition comprising titanium and clay and process therewith

(75) Inventor: Donald Edward Putzig, Newark, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. du Pont de Nemours and company, Wilmington, DE (US)

(21) Appl. No.: 12/012,583

(22) Filed: Feb. 4, 2008

Related U.S. Application Data
(62) Division of application No. 10/913,716, filed on Aug. 6, 2004.

(51) Int. Cl. B01J 31/12; C07C 67/39; C08G 63/16

(52) U.S. Cl. 528/279; 502/62; 560/96

(57) ABSTRACT

A composition and a process for producing an ester or polyester are provided. The composition comprises a titanium compound and clay or a dispersion of clay. The process comprises contacting a carbonyl compound, in the presence of a catalyst, with a glycol in which the catalyst comprises a titanium compound and clay or a dispersion of clay.

### FIELD OF THE INVENTION

The invention relates to composition that can be used as catalyst for producing polyester and a process using the composition for producing polyester wherein the composition comprises, or is produced from, a titanium compound and clay.

### BACKGROUND OF THE INVENTION

Polyesters such as, for example, polyethylene terephthalate, polytrimethylene terephthalate and polybutylene terephthalate, generally referred to as polyalkylene terephthalates, are a class of important industrial polymers. They are widely used in thermoplastic fibers, films, and molding applications.

Polyalkylene terephthalates can be produced by transesterification of a dialkyl terephthalate ester with a glycol followed by polycondensation or by direct esterification of terephthalic acid with the selected glycol followed by polycondensation. A catalyst is used to catalyze the esterification, transesterification and/or polycondensation.

Antimony, in the form of a glycol solution of antimony oxide, frequently is used as catalyst in the transesterification or esterification process. However, antimony forms insoluble antimony complexes that plug fiber spinnerets and leads in fiber spinning to frequent shutdowns to wipe spinnerets clean of precipitated antimony compounds. The antimony-based catalysts are also coming under increased environmental pressure and regulatory control, especially in food contact applications.

Organic titanates, such as tetraisopropyl and tetra n-butyl titanates, are known to be effective polycondensation catalysts for producing polyalkylene terephthalates in general, and frequently are the catalyst of choice. However, these catalysts tend to hydrolyze on contact with water, forming glycol-insoluble oligomeric species, which lose catalytic activity. These organic titanates may also generate a significant amount of yellow discoloration when used as polyesterification catalysts.

U.S. Pat. No. 4,705,764 discloses a process using silica, alumina, zeolite, a molecular sieve, or activated carbon as carrier for producing solid catalyst. JP 2000-327885A discloses mixing a swellable silicate (such as montmorillonite clay) and an ammonium salt (such as methyldodecyl ammonium chloride) in an aqueous medium.

There is always a need to develop an environmentally friendly catalyst, to improve the reactivity of a catalyst, and to reduce the yellowness of polyester product.

### SUMMARY OF THE INVENTION

A composition that can be used as a catalyst is provided, which comprises, or is produced from, an organic titanium compound, clay or a dispersion comprising clay, and optionally an alcohol.

A process that can be used for polycondensation of a carbonyl compound with a glycol, which comprises contacting, in the presence of the composition disclosed above.

### DETAILED DESCRIPTION OF THE INVENTION

The invention provides an organic titanium composition that can be suitable for use as an esterification, transesterification or polycondensation catalyst. The composition can comprise, consist essentially of, or consist of, an organic titanate, which can be in a solvent such as water or an alcohol or both, and clay or a dispersion comprising clay.

The titanium compound can be a titanium orthoester or a derivative thereof. The titanium compound can have the formula Ti(OR)₄ where each R is individually selected from an alkyl, cycloalkyl, alkaryl, hydrocarbyl radical containing from 1 to about 30, preferably 2 to about 18, and most preferably 2 to 12 carbon atoms per radical and each R can be the same or different. Examples of commercially available organic titanium compounds include, but are not limited to, TYZOR® TPT and TYZOR® TBT, (tetra isopropyl titanate and tetra n-butyl titanate, respectively), available from E. I. du Pont de Nemours and Company (“DuPont”), Wilmington, Del., U.S.A.

A titanium derivative can be a titanium chelate, which can be commercially available from, for example, DuPont or produced by any methods known to one skilled in the art. Examples of commercially available titanium chelates include, but are not limited to, acetylacetonate titanate chelate (TYZOR® AA), ethyl acetoacetate titanate chelate (TYZOR® DC), triethanolamine titanate (TYZOR® TE), and lactic acid titanate, ammonium salt (TYZOR® LA), all available from DuPont. Titanium chelate can refer to a compound having the formula of TiXₘ(OR)ₙ, where X is a radical derived from a chelating agent, m ranges from 0 and to 2 but not 0, and n ranges from 2 to 4; and R is the same as disclosed above. A chelating agent can be a carbonyl compound or an alkanolamine. A carboxylic acid, a ketone, an ester, a ketoester, a hydroxycarboxylic acid, an aminocarboxylic acid, triethanolamine, or combinations of two or more thereof can be used to produce the titanium chelates. Examples of suitable hydroxycarboxylic acids include, but are not limited to, lactic acid, glycolic acid, citric acid, tartaric acid, malic acid, and combinations of two or more thereof. Preferably the hydroxycarboxylic acid is an α-hydroxycarboxylic acid, in which the hydrocarbyl group or alkyl group has 1 to about 15, or 1 to 10 carbon atoms per group such as, for example, lactic acid.

For example, TYZOR® LA (titanium bis-ammonium lactate, a commercial solution containing 8.2 weight % titanium) disclosed above is an example of titanium hydroxycarboxylate, which is an aqueous solution with about 50% active ingredient, and is produced by reacting a titanium orthoester with lactic acid followed by neutralization with ammonia or ammonium hydroxide.

Any clay that, when used in combination with a titanium compound, can catalyze the polycondensation in polyester production can be used including natural clays such as smectite clays, synthetic clays, and modified clays. Natural clays include montmorillonite, saponite, hectorite, mica, vermiculite, bentonite, nontronite, beidellite, volkonskoite, margarite, pimelite, kyannite, kaolinite, halloysite, smectite, iolite, sepiolite, Fuller's earth, and combinations of two or more thereof. Synthetic clays include synthetic mica, synthetic saponite, synthetic hectorite, and combinations of two or more thereof. Modified clays include fluorinated montmorillonite, fluorinated mica, and combinations of two or more thereof. Most, if not all, clays are commercially available. Layered clays are an agglomeration of individual platelet particles that are closely stacked together like cards, in domains (tactoids). For example, clay having a layered clay material such as a smectite clay, which is in the form of a plurality of adjacent, bound layers, can be used. Generally, clay can have a surface area of from about 10 to about 500, or about 150 to about 300, or about 10 to about 100, or about 15 to about 50 m²/g. Individual platelet particles of the clays can have thickness of less than about 10 nm, or less than about 5 nm, or less than about 2 nm. The diameter can be in the range of about 1 to about 5000 nm or about 10 to about 3000 nm. These are in the form of essentially non-porous platelets. The platelets are typically swellable. Clay may be treated by a swelling agent to increase the spacing between platelet particles. If required, the particle size may be obtained by milling in a hammer mill, micronizer, wet mill, or other milling device.

Clay can be combined with a titanium compound in solid form or in dispersion form in a solvent such as water or alcohol or both. If in solid form, it can be combined with a solid, dispersed, solution, slurry, or combinations of two or more thereof, of titanium compound. A solution, dispersion, or slurry titanium compound generally is in a water or alcohol. The alcohol can be an alkylene glycol such as ethylene glycol. Description of alcohol (exchangeable with “glycol”) is disclosed below. The dispersion may be aided by the addition of a surface-active agent or dispersing agent.

Individual components can be combined in any order and the composition can be produced by any means known to one skilled in the art such as, mixing a blend of clay and titanium compound, at a temperature in the range of from about 0° C. to about 100° C., or about 20° C. to about 50° C.

Other compounds may be used with the titanium compound to modify catalyst performance, enhance solubility, prevent discoloration, or for other purposes. For example, zinc, cobalt or manganese may be used as a co-catalyst to enhance catalyst activity. Examples of suitable zinc salts include zinc acetate, zinc chloride, zinc nitrate, zinc sulfate, and combinations of two or more thereof. Examples of suitable cobalt salts include cobaltous acetate, cobaltous nitrate, cobaltous chloride, cobalt acetylacetonate, cobalt naphthenate, cobalt salicyl salicylate, and combinations of two or more thereof. Cobalt may be added also to act as color toner. Examples of suitable manganese salts include manganese benzoate, manganese chloride, manganese oxide, manganese acetate, manganese succinate, manganese acetyl acetonate, and combinations of two or more thereof. A phosphorus compound may be used to help control color formation. Still other compounds may be used to improve organic solubility, stability or for other reasons. Examples of catalyst combinations are disclosed, for example, in U.S. Pat. Nos. 6,066,714; 6,075,115; 6,080,834; 6,166,170; 6,255,441; and 6,303,738; descriptions of which are incorporated herein by reference. The amount of any of these compounds can be from 0 up to about half the weight of titanium used.

Also provided is a process for esterifying or polycondensing a carbonyl compound, in the presence of the composition disclosed above, with a glycol. The carbonyl compound can be (1) an aryl or alkyl dicarboxylic acid, (2) a salt thereof, (3) an ester thereof, (4) an oligomer thereof, or (5) combinations of two or more thereof. Aryl or alkyl means alkyl, alkenyl, aryl, alkaryl, aralkyl, or combinations of two or more thereof. For example, a reaction medium can comprise, consist essentially of, or consist of (1) a glycol and a dicarboxylate or (2) an oligomer having repeat units derived from a dicarboxylate. Dicarboxylate referred to here includes a dicarboxylic acid, an ester thereof, a salt thereof, or combinations of two or more thereof. The dicarboxylic acid can have the formula of HO₂CACO₂H in which A is an alkylene group, an arylene group, alkenylene group, or a combination of two or more thereof. Each A has about 2 to about 30, preferably about 3 to about 25, more preferably about 4 to about 20, and most preferably 4 to 15 carbon atoms per group. Examples of suitable dicarboxylic acids include, but are not limited to, terephthalic acid, isophthalic acid, napthalic acid, succinic acid, adipic acid, phthalic acid, glutaric acid, oxalic acid, maleic acid, and combinations of two or more thereof. The presently preferred dicarboxylic acid is terephthalic acid because the polyesters produced therefrom have a wide range of industrial applications. Examples of suitable esters include, but are not limited to, dimethyl phthalate, dimethyl terephthalate, dimethyl adipate and combinations of two or more thereof.

Examples of dicarboxylic acid metal salts or esters thereof includes compounds having the formula of (R¹O₂C)₂ArS(O)₂OM in which each R¹ can be the same or different and is hydrogen or an alkyl group containing 1 to about 6, preferably 2, carbon atoms. Ar is a phenylene group. M can be an alkali metal ion such as sodium or hydrogen. An example of the ester is bis-glycolate ester of 5-sulfo isophthalate sodium salt.

Any alcohol that can esterify an acid to produce an ester or polyester can be used in the present invention. The presently preferred glycol is an alkylene glycol of the formula (HO)ₙA¹ (OH)ₙ, in which A¹ has 2 to 30 carbon atoms per group and n is 1. Examples of suitable alcohol include, but are not limited to, ethylene glycol, propylene glycol, isopropylene glycol, butylene glycol, 1-methyl propylene glycol, pentylene glycol, diethylene glycol, triethylene glycol, and combinations of two or more thereof. The presently most preferred glycol is ethylene glycol or propylene glycol, for the polyesters produced therefrom have a wide range of industrial applications.

The composition can be used in producing esters, oligomers or polyesters by using any of the conventional melt or solid state techniques. The catalyst compositions are compatible with conventional esterification and transesterification catalysts (e.g., manganese, cobalt, and/or zinc salts) and may be introduced to the production process concurrent with, or following, introduction of the esterification catalyst. The catalyst compositions also have been found to be effective in promoting the esterification reaction, and may be used as a substitute for some or all of the esterification catalyst.

The contacting of carbonyl compound and alcohol in the presence of the catalyst can be carried out by any suitable means. For example, the carbonyl compound and glycol can be combined before being contacted with the catalyst, and may be reacted to form an oligomer. The oligomer may have a total of about 1 to about 100, preferably from about 2 to about 10 repeat units derived from the carbonyl compound and glycol. The catalyst can be first dissolved in a glycol by any suitable means such as mechanical mixing or stirring followed by combining the solution with (1) a carbonyl compound and (2) a glycol under a condition sufficient to effect the production of a ester, oligomer or polyester.

Any suitable condition to effect the production of an ester, oligomer or polyester can include a temperature in the range of from about 150° C. to about 500° C., preferably about 200° C. to about 400° C., and most preferably 250° C. to 300° C. under a pressure in the range of from about 0.001 to about 10 atmospheres for a time period of from about 0.1 to about 20 hours.

The molar ratio of the glycol to carbonyl compound can be any ratio so long as the ratio can effect the production of an ester, oligomer or polyester. Generally the ratio can be in the range of from about 1:1 to about 10:1, preferably about 1:1 to about 5:1, and most preferably 1:1 to 4:1.

The catalyst, expressed as Ti, can be present in the range of about 0.0001 to about 30,000, or about 0.001 to about 1,000, or 0.001 to 100, parts per million (ppm) by weight of the medium comprising the carbonyl compound and glycol. Other ingredients also can be present to enhance catalyst stability or performance.

A process for producing polyethylene terephthalate can be carried out by one of two routes: the transesterification of dimethyl terephthalate (DMT) with ethylene glycol followed by polycondensation, and the esterification of terephthalic acid (TPA) with ethylene glycol followed by polycondensation. In DMT-based technology, manganese can be used as transesterification catalyst. The amount is typically about 100 to 150 ppm, preferably about 120 ppm of Mn. When transesterification is complete, a phosphorus compound is often added to deactivate the manganese. The amount is typically about 50 ppm P. Then antimony (about 200 ppm) or titanium (about 20 ppm) is typically added for the polycondensation step. When using titanium, the catalyst composition of this invention may be used to reduce the amount of titanium used and improve the polyester color.

The catalyst composition disclosed here can be used in both processes. It may be added prior to esterification if there is a need to speed up this step. Any phosphorus needed may be added after esterification. The inventive catalyst composition can be added after esterification to the resulting oligomer. As with the DMT-based process, it can be used in the polycondensation step to eliminate the use of antimony or to reduce the amount of titanium and its related color problems. If necessary to improve the color, about 5 or 10 ppm of cobalt can be added to act as toner.

### EXAMPLES

The following Examples are provided to further illustrate the present invention and are not to be construed as to unduly limit the scope of the invention. TYZOR® TPT (tetra isopropyl titanate), obtained from DuPont, Wilmington, Del., USA was used.

Clay dispersion was made by adding montmorillonite KSP (obtained from Aldrich, Milwaukee, Wis., USA; surface area 20 to 40 m²/g) to water to a concentration of 1 to 5% by weight clay in water to produce a mixture, homogenizing (or using a Waring blender) the mixture to produce a homogenized mixture followed by filtering the homogenized mixture with a 125 mesh screen (US standard) to produce a filtrate, and adding ethylene glycol (equal to the volume of water) used to produce a glycol dispersion. Water was removed by either distillation or refluxing the glycol dispersion.

The process for producing terephthalic acid oligomer is illustrated as follows. An autoclave was charged with 100 pounds (45.4 Kg) of terephthalic acid and 67 pounds (30.4 Kg) of ethylene glycol. The batch was heated to 240° C. at an agitation speed of 15 rpm, and 21.6 lbs. (9.8 Kg) of water and 14.3 lbs. (6.5 Kg) of ethylene glycol were removed. The charge was then heated to 275° C. over the course of 90 minutes, and the remaining ethylene glycol was removed at 285° C. and below 2 mm Hg vacuum (267 Pa). Once the condensation mass was judged to be complete, the molten mass was extruded into an aqueous bath to solidify the product. The resultant oligomer was dried to remove residual moisture before use.

A 1-liter resin kettle was provided with a Jiffy Mixer agitator rotating at 40 rpm, a thermocouple, condenser and nitrogen sweep. To this kettle was added the catalyst to be tested, 115 ml of ethylene glycol, and 400 g of terephthalic acid oligomer prepared above. The agitator was turned on and the temperature was increased to 275° C. over a period of about 2.5 hours. The contents were polymerized by holding under agitation at 275° C. and a pressure of 120 mm Hg (16 kPa) for 20 minutes, and at 280° C. and a pressure of 30 mm Hg (4 kPa) for an additional 20 minutes. The contents were then held under agitation at 285° C. at 1 to 2 mm Hg pressure for a time sufficient to reach 15 ounce-inch (0.106 Newton-meter) torque as measured by an Electro-Craft Motomatic torque controller. The time for this step was recorded as the Finish Time, and varied with the catalyst used. The polymer melt was then poured into a water bath to solidify the melt, and the resultant solid annealed at 150° C. for 12 hours and ground to pass through a 2 mm filter for color measurements using the previously-described spectrophotometer. Results comparing the color as measured spectrophotometrically are given in Table 2 below.

Color of the resulting oligomer and any polymer produced therefrom was measured in terms of the L-value and b-value, using an instrument such as the SP-78 Spectrophotometer. The L-value shows brightness, with the greater the numerical value showing higher (desirable) brightness. A value of 78 or more would be considered good. It will vary with additives such as cobalt. The b-value shows the degree of yellowness, with a higher numerical value showing a higher (undesirable) degree of yellowness. For the laboratory trials, b-values below 7 were considered a success. The a-value represents degree of redness: a higher positive a-value is redder; a lower negative a-value is greener.

The following tables are results of using a 5% clay dispersion in ethylene glycol to a final concentration shown in the tables (loading values in ppm (parts per million by weight). The oligomer used was based on terephthalic acid and ethylene glycol using no catalyst for the oligomerization step. PET refers to polyethylene terephthalate.

TABLE 1
PET using Antimony and Clay Catalysts

| | | | | | Time | Temp |
|---|---|---|---|---|---|---|
| Run# | L-color | a-color | b-color | Sb/Clay (ppm) | (min) | (C.) |
| 1 | 83.18 | −1.55 | 4.9 | 200/0 | 110 | 285 |
| 2 | 81.63 | −0.42 | 4.75 | 0/500 | 140 | 285 |
| 3 | 78.8 | 0.02 | 5.63 | 0/500 | 180 | 285 |
| 4 | 78.31 | −1.21 | 4.43 | 200/1000 | 115 | 285 |

Table 1 shows that using 500 ppm clay as catalyst had much lower activity than using 200 ppm Sb alone as catalyst. Combining 200 ppm Sb and 1000 clay did not improve the catalytic activity (in terms of reaction time). The b color of the polyester produced using clay was similar to that using Sb.

TABLE 2
PET using Titanium and Clay Catalysts

| | | | | | Time | Temp |
|---|---|---|---|---|---|---|
| Run# | L-color | a-color | b-color | Ti/Clay (ppm) | (min) | (C.) |
| 21 | 81.34 | −1.64 | 6.24 | 10/0 | 125 | 285 |
| 22 | 80.36 | −0.79 | 5.47 | 5/1000 | 110 | 285 |
| 23 | 82.78 | −0.49 | 6.55 | 10/1000 | 75 | 285 |

Table 2 shows that, replacing 200 ppm Sb with 10 ppm Ti, 1000 ppm clay improved the catalytic activity significantly (reaction time shortened from 115 minutes in Table 1 to merely 75 minutes) and the b color remained satisfactory.

TABLE 3
PET using Titanium, Cobalt, Zinc and Clay Catalysts

| | | | | | Time | Temp |
|---|---|---|---|---|---|---|
| Run# | L-color | a-color | b-color | Ti/Co/Zn/Clay (ppm) | (min) | (C.) |
| 31 | 76.67 | −1.51 | 7.02 | 10/5/20/0 | 75 | 285 |
| 32 | 79.16 | −0.95 | 5.38 | 10/5/20/1000 | 60 | 285 |
| 33 | 79.16 | −0.38 | 9.5 | 10/0/45/0 | 120 | 285 |
| 34 | 82.28 | −0.44 | 5.38 | 10/0/45/1000 | 50 | 285 |

Table 3 shows that addition of cocatalyst Zn or toner Co to the Ti/clay composition further improved the catalytic activity (faster reaction rate) and the product b color.

TABLE 4
PET using Titanium, Cobalt Zinc and
Clay Catalysts vs Temperature

| | | | | | Time | Temp |
|---|---|---|---|---|---|---|
| Run | L-color | a-color | b-color | Ti/Co/Zn/Clay(ppm) | (min) | (C.) |
| 40 | 81.47 | −0.67 | 6.06 | 10/5/45/1000 | 60 | 285 |
| 41 | 77.4 | −0.94 | 6.81 | 10/5/45/1000 | 45 | 295 |
| 42 | 83.47 | −0.47 | 5.57 | 10/5/45/1000 | 85 | 275 |

Table 4 shows that PET produced at a higher temperature had slightly poorer b colors. However, reaction time improved.

A solid montmorillonite was also added to the reaction medium at similar final concentration of clay. It showed that the finishing time was 125 minutes for a 10 ppm Ti/1000 ppm clay test. The product had an L color of 85.3, a-color of −0.73 and b-color of 6.89. This demonstrates that a solid clay can be used in combination with a titanium compound. In a separate run, using montmorillonite K-10 (surface area 220-270 m²/g), it showed a finishing time of 100 minutes for a 10 ppm Ti/1000 ppm clay test. The b color of the product for this run, however, was significantly better at 3.68 units (L=81.39 and a=−0.98).

1-14. (canceled)

15. A process to produce an organic titanium composition comprising contacting a titanium compound with clay at a temperature in the range of 0° C. to 100° C. to produce a composition, wherein said titanium compound is in a liquid form in water or an alcohol or both and has the formula of Ti(OR)₄ or TiXₘ(OR)ₙ; each R is independently an alkyl radical, cycloalkyl radical, alkaryl radical, or combinations of two or more thereof containing from 1 to about 30 carbon atoms per radical; X is a radical derived from a chelating agent, m ranges from 0 and to 2 but not 0, and n ranges from 2 to 4, and said clay is in dry form or dispersed in an alcohol.

16. A process according to claim 15 wherein said alcohol is ethylene glycol.

17. A process according to claim 16 wherein said clay is dispersed in said alcohol.

18. A process according to claim 17 wherein said titanium compound is tetraisopropyl titanate, tetra-n-butyl titanate, titanium bis-ammonium lactate, or combinations of two or more thereof and said clay is montmorillonite.

19. A process according to claim 17 wherein said wherein said clay is montmorillonite, saponite, hectorite, mica, vermiculite, bentonite, nontronite, beidellite, volkonskoite, margarite, pimelite, kyannite, kaolinite, halloysite, smectite, iolite, sepiolite, Fuller's earth, synthetic mica, synthetic saponite, synthetic hectorite, fluorinated montmorillonite, fluorinated mica, or a combination of two or more thereof, said titanium compound is tetraisopropyl titanate, tetra-n-butyl titanate, titanium bis-ammonium lactate, or combinations of two or more thereof; and wherein said clay has a surface area of about 150 to about 300 m²/g, a thickness of less than about 2 nm, and a diameter in the range of from about 10 to about 3000 nm.

20. A process according to claim 18 wherein said wherein said clay has a surface area of about 15 to about 50 m², a thickness of less than about 2 nm, and a diameter in the range of from about 10 to about 3000 nm.

21. A process according to claim 16 wherein said clay is in dry form.

22. A process according to claim 21 wherein said titanium compound is tetraisopropyl titanate, tetra-n-butyl titanate, titanium bis-ammonium lactate, or combinations of two or more thereof and said clay is montmorillonite.

23. A process according to claim 21 wherein said clay is montmorillonite, saponite, hectorite, mica, vermiculite, bentonite, nontronite, beidellite, volkonskoite, margarite, pimelite, kyannite, kaolinite, halloysite, smectite, iolite, sepiolite, Fuller's earth, synthetic mica, synthetic saponite, synthetic hectorite, fluorinated montmorillonite, fluorinated mica, or a combination of two or more thereof; said titanium compound is tetraisopropyl titanate, tetra-n-butyl titanate, titanium bis-ammonium lactate, or combinations of two or more thereof; and wherein said clay has a surface area of about 150 to about 300 m²/g, a thickness of less than about 2 nm, and a diameter in the range of from about 10 to about 3000 nm.

24. A process according to claim 21 wherein said titanium compound is tetraisopropyl titanate, tetra-n-butyl titanate, titanium bis-ammonium lactate, or combinations of two or more thereof and said clay is montmorillonite and has a surface area of about 15 to about 50 m²/g, a thickness of less than about 2 nm, and a diameter in the range of from about 10 to about 3000 nm.

25. A process comprising contacting, in the presence of a catalyst composition, a carbonyl compound with a glycol under a condition effective to produce an ester or polyester wherein said composition is is prepared by a process comprising contacting a titanium compound with clay at a temperature in the range of 0° C. to 100° C. to produce a composition, wherein said titanium compound is in a liquid form in water or an alcohol or both and has the formula of Ti(OR)₄ or TiXₘ(OR)ₙ, each R is independently an alkyl radical, cycloalkyl radical, alkaryl radical, or combinations of two or more thereof containing from 1 to about 30 carbon atoms per radical, X is a radical derived from a chelating agent, m ranges from 0 and to 2 but not 0, and n ranges from 2 to 4, and said clay is in dry form or dispersed in an alcohol.

26. A process according to claim 25 wherein said carbonyl compound is terephthalic acid, dimethyl terephthalate, an oligomer of terephthalic acid, or combinations of two or more thereof and said glycol is ethylene glycol.

27. A process according to claim 25 wherein said titanium compound is tetraisopropyl titanate, tetra-n-butyl titanate, titanium bis-ammonium lactate, or combinations of two or more thereof and said clay is montmorillonite.

28. A process according to claim 26 wherein said clay is montmorillonite, saponite, hectorite, mica, vermiculite, bentonite, nontronite, beidellite, volkonskoite, margarite, pimelite, kyannite, kaolinite, halloysite, smectite, iolite, sepiolite, Fuller's earth, synthetic mica, synthetic saponite, synthetic hectorite, fluorinated montmorillonite, fluorinated mica, or a combination of two or more thereof; said titanium compound is tetraisopropyl titanate, tetra-n-butyl titanate, titanium bis-ammonium lactate, or combinations of two or more thereof; and wherein said clay has a surface area of about 150 to about 300 m²/g, a thickness of less than about 2 nm, and a diameter in the range of from about 10 to about 3000 nm.

29. A process according to claim 26 wherein said titanium compound is tetraisopropyl titanate, tetra-n-butyl titanate, titanium bis-ammonium lactate, or combinations of two or more thereof and said clay is montmorillonite and has a surface area of about 15 to about 50 m²/g, a thickness of less than about 2 nm, and a diameter in the range of from about 10 to about 3000 nm.

30. A process according to claim 26 wherein said titanium compound is tetraisopropyl titanate, tetra-n-butyl titanate, titanium bis-ammonium lactate, or combinations of two or more thereof and said clay is montmorillonite, wherein said alcohol is an alkylene glycol.

31. A process according to claim 26 wherein said titanium compound is tetraisopropyl titanate, tetra-n-butyl titanate, titanium bis-ammonium lactate, or combinations of two or more thereof, said clay is montmorillonite and has a surface area of about 15 to about 50 m²/g, a thickness of less than about 2 nm, and a diameter in the range of from about 10 to about 3000 nm, said alcohol is ethylene glycol.

32-34. (canceled)
